(19) United States
(12) Patent Application Publication
(10) Pub. No.: US 2015/0080252 A1
(43) Pub. Date: Mar. 19, 2015

(54) GENE EXPRESSION SIGNATURES ASSOCIATED WITH RESPONSE TO IMATINIB MESYLATE IN GASTROINTESTINAL STROMAL TUMORS AND USE THEREOF FOR PREDICTING PATIENT RESPONSE TO THERAPY AND IDENTIFICATION OF AGENTS WHICH HAVE EFFICACY FOR THE TREATMENT OF CANCER

(71) Applicant: Fox Chase Cancer Center, Jenkintown, PA (US)

(72) Inventors: Andrew K. Godwin, Leawood, KS (US); Burton Eisenberg, Woodstock, VT (US); Yuliya Skorobogatko, Jenkintown, PA (US); Lori Rink, Willow Grove, PA (US); Andrew Kossenkov, Huntingdon Valley, PA (US); Michael F. Ochs, Oreland, PA (US)

(21) Appl. No.: 14/467,984

(22) Filed: Aug. 25, 2014

Related U.S. Application Data
(63) Continuation of application No. 13/265,686, filed on Dec. 8, 2011, now abandoned.
(60) Provisional application No. 61/171,297, filed on Apr. 21, 2009.

(51) Int. Cl. C12Q 1/68

(52) U.S. Cl. CPC C12Q 1/6886; C12Q 2600/158; C12Q 2600/16
USPC 506/9; 506/17

(57) ABSTRACT

Compositions and methods are disclosed for identifying agents useful for the treatment of malignancy, particularly GISTs which are resistant to imatinib mesylate (IM). In a preferred embodiment, agents which sensitize cancer cells to IM are provided.

This application is a continuation of U.S. patent application Ser. No. 13/256,686 filed Dec. 8, 2011, which is a §371 filing of PCT/US10/31883 filed Apr. 21, 2010 which in turn claims priority to U.S. Provisional Application 61/171,297 filed Apr. 21, 2009, the entire contents of each being incorporated herein by reference as though set forth in full.

This invention was made with government support under Grant Numbers CA106588, U10 CA21661, P30 CA006927, LM009382, and CA0090035-31 awarded by the National Institutes of Health. The government has certain rights in the invention.

### FIELD OF THE INVENTION

This invention relates to the fields of oncology and medicine. More specifically, the invention provides biomarkers and methods of use thereof which aid the clinician in identifying those patients most likely to benefit from certain treatment regimens. The markers disclosed herein are also useful in assays to identify therapeutic agents useful for the treatment of malignancy.

### BACKGROUND OF THE INVENTION

Several publications and patent documents are cited throughout the specification in order to describe the state of the art to which this invention pertains. Each of these citations is incorporated by reference herein as though set forth in full.

Gastrointestinal stromal tumors (GISTs) are the most common mesenchymal tumors of the digestive tract, with between 3,300 to 6,000 new cases diagnosed each year in the US (1). The most common primary sites for these neoplasms are the stomach (60-70%) (2, 3), followed by the small intestine (25-35%) (4, 5), and to a much lesser degree the colon and rectum (10%) (6). GISTs have also been observed in the mesentery, omentum, esophagus, and the peritoneum (2, 7). GISTs occur most frequently in patients over 50, with a median age of presentation of 58 years; however, GISTs have also been observed in the pediatric population (8). These tumors contain smooth muscle and neural elements as described originally by Mazur and Clark in 1983, and are thought to arise from the interstitial cells of Cajal (9, 10). GISTs express and are clinically diagnosed by immunohistochemical staining of the 145 kDa transmembrane glycoprotein, KIT, by the CD117 antibody. The majority (˜70%) of GISTs possess gain-of-function mutations in c-KIT in either exons 9, 11, 13 or 17, causing constitutive activation of the kinase receptor, whereas smaller subsets of GISTs possess either gain-of-function mutations in PDGFRA (exons 12, 14, or 18) (˜10%) or no mutations in either KIT or PDGFRA and are therefore referred to as wild-type (WT) GISTs (˜15-20%) (11-14). The primary treatment for GIST is surgical resection, which is often not curative in high risk GIST due to a high incidence of reoccurrence (15, 16). Since 2002, IM, an oral 2-phenylaminopyrimidine derivative that works as a selective inhibitor against mutant forms of type III tyrosine kinases such as KIT, PDGFRA, and BCR/ABL, has become a standard treatment for patients with metastatic and/or unresectable GIST, with objective responses or stable disease obtained in >80% of patients (17, 18). Response to IM has been correlated to the genotype of a given tumor (14). GIST patients with exon 11 KIT mutations have the best response and disease-free survival, while other KIT mutation types and WT GIST have worse prognoses. Despite the efficacy of IM, some patients experience primary and/or secondary resistance to the drug. [¹⁸F] fluorodeoxyglucose-positron emission tomography (PET) can be used to rapidly assess tumor response to IM (19); however, there are cases in which GISTs do not take up significant amounts of the glucose precursor and therefore this scanning method is of questionable value in evaluating response in this group of patients. Strategies for treatment of progressive disease can include: IM dose escalation (20), IM in combination with surgery, and alternative KIT/PDGFRA inhibitors including: sunitinib (21). There are also options to participate in clinical trials evaluating nilotinib (22), dasatinib (23) and HSP90 inhibitors (24). What may eventually prove to be the most effective paradigm in the clinical management of GIST is the development of individualized treatment approaches based on KIT and PDGFRA mutational status and/or predictive gene signatures of drug response. Ideally, in the future patients may be pre-selected for treatment with IM or additional first and second line therapies based on these tumor specific response markers.

### SUMMARY OF THE INVENTION

In accordance with the present invention, a method of identifying patients likely to benefit from treatment of GIST with imatinib mesylate (IM) is provided. An exemplary method comprises providing a genetic signature of differentially expressed nucleic acids which provide a prognostic indicator of response to IM treatment; isolating a plurality of nucleic acids from a patient sample; and assessing expression levels of at least one nucleic acid gene product from those listed in Table 2, wherein an increase in the expression level of said at least one gene product in said patient relative to expression levels of observed in the genetic signature associated with response to IM, is indicative of an increase in resistance to IM therapy. In one embodiment the patient has refractory GIST.

In another aspect of the invention, a method for inhibiting development of imatinib mesylate (IM) resistance in a subject in need thereof is disclosed. An exemplary method comprises administering an effective amount of a pharmaceutical composition comprising a therapeutic amount of an agent which inhibits expression or activity of at least one target gene selected from the group listed in Table 2. Inhibition of the target gene being correlated with reduced resistance to IM treatment in said cancer cell in said subject. In a preferred embodiment the cancer is a GIST. In another embodiment, the agent is at least one siRNA which is effective to down modulate expression of at least one KRAB domain containing zinc finger transcriptional repressor, such as those siRNAs provided in SEQ ID NOS: 1-18. In alternative embodiments, the agent may be a peptide, a small molecule or other type of inhibitory nucleic acid.

In another aspect of the invention, a method for identifying agents which increase the sensitivity of a cancer cell to imatinib mesylate (IM) or sunitnib is provided. An exemplary method entails providing a sample of cancer cells which have lost sensitivity to IM treatment; incubating the IM resistant cells in the presence and absence of an agent which effectively down modulates expression of at least one gene product selected from the group listed in Table 2; contacting the cells with IM and/or sunitinib and assessing whether sensitivity is restored in cells treated with the agent relative to non-treated control cells, thereby identifying an agent which sensitizes cancer cells to IM and/or sunitinib. Preferably the cells are GIST cells and the agent is an siRNA which inhibits expression of at least one ZNF family member.

### BRIEF DESCRIPTION OF THE DRAWINGS

FIGS. 1A and 1B. RTOG-S0132 trial design and patient response to IM. FIG. 1A) Patients with primary or recurrent operable GISTs were screened for KIT (CD 117) expression by IHC for eligibility. Prior to IM treatment, a CT was performed and biopsies were collected by core needle aspiration. Patients were then treated with an 8-12 week regimen of IM, followed by cyto-reductive surgery. A CT was also performed once during treatment (˜4-6 weeks into IM treatment) and immediately prior to surgery. FIG. 1B) (Top) Percentage of tumor growth based on CT measurements taken from the longest cross sectional diameter of the primary GIST or the index metastatic lesion(s) for each RTOG-S0132 patient. (Bottom) Specific samples (pre-, post- or both) used for microarray analysis classified as Group A or B based on the percent of tumor shrinkage/growth visualized by CT. Mutational analysis of most patients was performed and is denoted by color of bar (white=KIT exon 11 mutants, grey=wild-type GISTs, dark grey=KIT exon 9 mutants, light grey=not enough DNA available for mutational analysis). Group A is defined as ≧25% tumor shrinkage after 8-12 weeks of IM and Group B contains tumors demonstrating <25% tumor reduction, no change, or evidence of tumor enlargement after 8-12 weeks of IM.

FIG. 2. Gene expression profiles associated with response to IM. A heat map showing the HSA19p12-13.1 KRAB-ZNF hierarchical cluster. In the image blue represents down-regulation, whereas red represents up-regulation. Patients who initially responded rapidly to IM clearly show decreased KRAB-ZNF expression compared to the others.

FIGS. 3A and 3B. KRAB-ZNF gene expression on chromosome 19p12-13.1 before and after IM therapy. FIG. 3A: Analysis of pre-treatment ratios of tumors showing >25% (Group A) or <25 reduction (Group B) using data from 28 patients for all genes in the 19p12-13.1 locus. All genes, in this locus (red box) showed higher mean ZNF expression levels in Group B samples (i.e. lower Group A/Group B ratio) while adjoining genes showed roughly equal expression between the two groups. FIG. 3B: Analysis of changes in expression of genes in this locus upon IM treatment in Group B samples with >70% tumor cellularity. Red bars represent means of pre-treatment samples from Group B and blue bars represent means of post-treatment samples from Group B.

FIG. 4. Validation of ZNF Gene Expression by qRT-PCR. Fold expression changes of three of the ZNFs within the predictive signature gene panel, i.e., ZNF 43, ZNF 208 and ZNF 91, were measured using qRT-PCR. The ratios of each gene to control (HPRT or actin) were measured using total RNAs from nine pretreatment samples (5 in Group A and 4 in Group B) and universal human reference RNA. The relative median mRNA levels for ZNF 43, ZNF 208, and ZNF 91 in Group A were 412-, 257- and 77-fold higher as compared to controls, whereas the median levels in Group B were 21-, 18-, and 11-fold normalized to controls, respectively. Two-sided Wilcoxon rank sum tests were used to compare the distribution of ZNF 43, ZNF 208, and ZNF 91 mRNA expression between the two groups and Pearson's coefficients were used to measure the pairwise correlation of the ZNF gene expression. Tests were conducted using a 5% type I error. The predictive value of ZNF 43 and ZNF 208 were found to be statistically significant (*p=0.02). Results are representative of three independent experiments.

FIG. 5. Heatmap showing sensitizing index (SI) ranging from 0.6 (blue) to 1.16 (yellow). Sensitization was tested in GIST-T1 cells in the presence of four different drugs including: Ifosfamide (top row), doxorubicin (second row), sunitinib (third row) and imatinib (bottom row). Dark grey indicates those genes which are “sensitizing hits” that have <0.85 ratio of drug/vehicle, white indicates those genes that were not “sensitizing hits”.

FIG. 6. Quantitative RT-PCR analysis showing efficient knockdown of the targets of interest relative to siCON (scrambled, non-targeting siRNA) in GIST-T1 cells.

### DETAILED DESCRIPTION OF THE INVENTION

Despite initial efficacy of imatinib mesylate (IM) in most gastrointestinal stromal tumor (GIST) patients, many experience primary and secondary drug resistance. Therefore, clinical management of GIST may benefit from further molecular characterization of tumors before and after IM treatment. The question of whether IM can be safe and effective as a rapid cytoreductive agent if administered prior to surgical resection has been evaluated in a recent novel Phase II trial (Radiation Therapy Oncology Group Study 0132) of 8 to 12 weeks of neoadjuvant followed by adjuvant IM for either locally advanced primary or metastatic operable GIST. In this study, biopsies were taken at time of enrollment, patients were treated with IM for 8 to 12 weeks prior to resection, followed by adjuvant IM treatment for 2 years. Contrast enhanced CT scans were performed before, 4-6 weeks into treatment, and after the neoadjuvant IM regimen in order to document classic tumor response by RECIST criteria. Based on CT response data, patients for this study were classified into two groups: Group A (defined as ≧25% tumor shrinkage after 8-12 weeks of IM) and Group B (<25% tumor shrinkage, unchanged, or evidence of tumor enlargement after 8-12 weeks of IM). Microarray analysis of pre-treatment GIST biopsies identified a gene signature of 38 response genes. These included Kruppel-associated box (KRAB)-zinc finger (ZNF) genes that were significantly expressed in tumor biopsies from patients less responsive to short-term treatment of imatinib.

Based on SAM analysis (FDR=10%), thirty-eight genes were expressed at significantly lower levels in the pre-treatment samples of those tumors that significantly responded to 8 to 12 weeks of IM, i.e., >25% tumor reduction. Eighteen of these genes encoded KRAB domain containing zinc finger (KRAB-ZNF) transcriptional repressors. Importantly, ten KRAB-ZNF genes mapped to a single locus on chromosome 19p, and a subset of these predicted likely response to IM-based therapy in a naïve panel of GISTs. Furthermore, we found that modifying expression of genes within this predictive signature can enhance the sensitivity of GIST cells to IM.

Using clinical samples from a prospective neoadjuvant phase II trial we have identified a gene signature which includes KRAB-ZNF 91 subfamily members that (4) may be both predictive of and functionally associated with likely response to short term IM treatment.

### DEFINITIONS

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

An “imatinib mesylate (IM) sensitivity marker (ISM)” is a marker which is associated differential sensitivity to IM (Gleevac). Such markers may include, but are not limited to, nucleic acids, proteins encoded thereby, or other small molecules. See Table 2. These markers can be used to advantage to identify those patients likely to respond to IM therapy from those that are unlikely to respond. They can also be targeted to modulate the response to IM therapy or used in screening assays to identify agents that have efficacy for the treatment and management of GIST.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with benign breast disease. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form. By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any IM sensitivity marker gene or nucleic acid, but does not hybridize to other nucleotides. Also polynucleotide which “specifically hybridizes” may hybridize only to a IM sensitivity marker shown in the Tables contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the IM sensitivity marker nucleic acid molecule such that the latter is transcribed into mRNA. Host cell machinery then translates mRNA into a polypeptide. The skilled artisan is aware of many other suitable promoter elements which can be used in the vectors of the invention.

Those skilled in the art will recognize that a nucleic acid vector can contain nucleic acid elements other than the promoter element and the IM sensitivity marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably a IM sensitivity marker molecule, such as a marker shown in the table provided below. Samples may include but are not limited to cells, body fluids, including blood, serum, plasma, urine, saliva, tears, pleural fluid and the like.

### Methods of Using IM Sensitivity (IMS) Associated Markers

IMS marker containing nucleic acids, including but not limited to those listed below may be used for a variety of purposes in accordance with the present invention. IMS associated marker containing DNA, RNA, or fragments thereof may be used as probes to detect the presence of and/or expression of IMS markers. Methods in which IMS marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

Further, assays for detecting IMS markers may be conducted on any type of biological sample, including but not limited to body fluids (including blood, urine, serum, gastric lavage), any type of cell (such as brain cells, white blood cells, mononuclear cells) or body tissue.

From the foregoing discussion, it can be seen that IMS marker containing nucleic acids, vectors expressing the same, IMS marker proteins and anti-IMS specific marker antibodies of the invention can be used to detect IMS associated molecules in body tissue, cells, or fluid, and alter IMS marker protein expression for purposes of assessing the genetic and protein interactions involved in the development of IM sensitivity and the development of resistance thereto.

In most embodiments for screening for IMS markers, the IMS marker containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art.

Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus any of the aforementioned techniques may be used to detect or quantify IMS marker expression and accordingly, predict a patients likelihood of benefiting from IM administration for the treatment of GIST.

### Kits and Articles of Manufacture

Any of the aforementioned products can be incorporated into a kit which may contain a IMS marker polynucleotide or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, means for obtaining a biopsy or cell sample, suitable culturing reagents, a vessel for administration, an assay substrate, or any combination thereof.

### Methods of Using IMS Markers for Development of Therapeutic Agents

Since the markers identified herein have been associated with the development of IM resistance, methods for identifying agents that modulate the activity of the genes and their encoded products should result in the generation of efficacious therapeutic combinations of agents for the treatment of a variety of proliferative disorders including the management of GIST.

The genes listed in Table 2 contain regions which provide suitable targets for the rational design of therapeutic agents which modulate their activity. Small peptide molecules, inhibitory RNAs, and chemical compounds having affinity for these regions may be used to advantage in the design of therapeutic agents which effectively modulate the activity of the encoded proteins.

Molecular modeling should facilitate the identification of specific organic molecules with capacity to bind to the active site of the proteins listed in Table 2 based on conformation or key amino acid residues required for function. A combinatorial chemistry approach will be used to identify molecules with greatest activity and then iterations of these molecules will be developed for further cycles of screening. In certain embodiments, candidate agents can be screening from large libraries of synthetic or natural compounds. Such compound libraries are commercially available from a number of countries including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, the can be formulated in to pharmaceutical compositions and utilized for the treatment of malignancy.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered IMS marker gene. These host cell lines or cells are defective at the polypeptide level. The host cell lines or cells are grown in the presence of drug compound. The rate of cellular proliferation and transformation of the host cells is measured to determine if the compound is capable of regulating the proliferation and transformation of the defective cells.

The test compounds used in the methods can be obtained using any of the numerous approaches in the art including combinatorial library methods as mentioned above, including: biological libraries; peptoid libraries (libraries of molecules having the functionalities of peptides, but with a novel, non-peptide backbone which are resistant to enzymatic degradation but which nevertheless remain bioactive; e.g., Zuckermann et al. (1994) J. Med. Chem. 37:2678); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the literature, for example in: DeWitt et al., Proc. Natl. Acad. Sci. USA, 90:6909, 1993; Erb et al., Proc. Natl. Acad. Sci. USA, 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl., 33:2061, 1994; and Gallop et al., J. Med. Chem., 37: 1233, 1994. Libraries of compounds may be presented in solution (e.g., Houghten, Bio/Techniques, 13:412421, 1992), or on beads (Lam, Nature, 354:82-84, 1991), chips (Fodor, Nature 364:555-556, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA, 89:1865-1869, 1992) or phage (Scott and Smith, Science, 249:386-390, 1990; Devlin, Science, 249:404-406, 1990; Cwirla et al., Proc. Natl. Acad. Sci. USA, 87:6378-6382, 1990; and Felici, J. Mol. Biol., 222:301-310, 1991).

In one embodiment, a cell-based assay is employed in which a cell that expresses a target protein or biologically active portion thereof is contacted with a test compound. The ability of the test compound to modulate expression or activity of the target protein is then determined.

The ability of the test compound to bind to a target protein or modulate target protein binding to a compound, e.g., a target protein substrate, can also be evaluated. This can be accomplished, for example, by coupling the compound, e.g., the substrate, with a radioisotope or enzymatic label such that binding of the compound, e.g., the substrate, to the target protein can be determined by detecting the labeled compound, e.g., substrate, in a complex. Alternatively, the target protein can be coupled with a radioisotope or enzymatic label to monitor the ability of a test compound to modulate target protein binding to a target protein substrate in a complex. For example, compounds (e.g., target protein substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a target protein substrate) to interact with target protein with or without the labeling of any of the interactants can be evaluated. For example, a microphysiometer can be used to detect the interaction of a compound with a target protein without the labeling of either the compound or the target protein (McConnell et al., Science 257:1906-1912, 1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between a compound and a target protein.

In yet another embodiment, a cell-free assay is provided in which a target protein or biologically active portion thereof is contacted with a test compound and the ability of the test compound to bind to the target protein or biologically active portion thereof is evaluated. In general, biologically active portions of target proteins to be used in assays described herein include fragments that participate in interactions with other molecules, e.g., fragments with high surface probability scores. Cell-free assays involve preparing a reaction mixture of the target protein and the test compound under conditions and for a time sufficient to allow the two components to interact and bind, thus forming a complex that can be removed and/or detected Another approach entails the use of phage display libraries engineered to express fragment of the polypeptides encoded by at least one of the genes listed in Table 2 on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of marker containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

### Pharmaceuticals and Peptide Therapies

The elucidation of the role played by the IMS markers described herein in the resistance of certain GIST cases to IM administration facilitates the development of pharmaceutical compositions useful for treatment and diagnosis of this disease. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

This invention further pertains to novel agents identified by the above-described screening assays. Accordingly, it is within the scope of this invention to further use an agent (compound) identified as described herein (e.g., a target protein modulating agent, an siRNA, a target protein-specific antibody, or a target protein-binding partner) in an appropriate animal model to determine the efficacy, toxicity, side effects, or mechanism of action, of treatment with such an agent. Furthermore, novel agents identified by the above-described screening assays can be used for treatments as described herein.

For example, molecules that are targeted to a target RNA are useful for the methods described herein, e.g., inhibition of target protein expression, e.g., for treating IM resistance GIST. Examples of nucleic acids include siRNAs described further hereinbelow. Other such molecules that function using the mechanisms associated with RNAi can also be used including chemically modified siRNAs and vector driven expression of hairpin RNA that are then cleaved to siRNA. The nucleic acid molecules or constructs that are useful as described herein include dsRNA (e.g., siRNA) molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, can transcribed be in vitro from a DNA template, or can be transcribed in vivo from, e.g., shRNA. The dsRNA molecules can be designed using methods known in the art, e.g., Dharmacon.com (see, siDESIGN CENTER) or “The siRNA User Guide,” available on the Internet.

Negative control siRNAs (“scrambled”) generally have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. Controls can also be designed by introducing an appropriate number of base mismatches into the selected siRNA sequence.

The nucleic acid compositions that are useful for the methods described herein include both siRNA and crosslinked siRNA derivatives. Crosslinking can be used to alter the pharmacokinetics of the composition, for example, to increase half-life in the body. Thus, the invention includes siRNA derivatives that include siRNA having two complementary strands of nucleic acid, such that the two strands are crosslinked. For example, a 3′OH terminus of one of the strands can be modified, or the two strands can be crosslinked and modified at the 3′OH terminus. The siRNA derivative can contain a single crosslink (e.g., a psoralen crosslink). In some cases, the siRNA derivative has at its 3′ terminus a biotin molecule (e.g., a photocleavable biotin), a peptide (e.g., a Tat peptide to facilitate cellular uptake), a nanoparticle, a peptidomimetic, organic compounds (e.g., a dye such as a fluorescent dye), or dendrimer. Modifying SiRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions described herein can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished using methods known in the art, e.g., using the methods of Lambert et al., Drug Deliv. Rev., 47, 99-112, 2001 (describes nucleic acids loaded to polyalkylcyanoacrylate (PACA) nanoparticles); Fattal et al., J. Control Release, 53:137-143, 1998 (describes nucleic acids bound to nanoparticles); Schwab et al., Ann. Oncol., 5 Suppl. 4:55-8, 1994 (describes nucleic acids linked to intercalating agents, hydrophobic groups, polycations or PACA nanoparticles); and Godard et al., Eur. J. Biochem., 232:404-410, 1995 (describes nucleic acids linked to nanoparticles).

The nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the molecule can be radiolabeled, e.g., using H, P, or other appropriate isotope.

Synthetic siRNAs can be delivered into cells by cationic liposome transfection and electroporation. Sequences that are modified to improve their stability can be used. Such modifications can be made using methods known in the art (e.g., siSTABLE™, Dharmacon). Such stabilized molecules are particularly useful for in vivo methods such as for administration to a subject to decrease target protein expression. Longer term expression can also be achieved by delivering a vector that expresses the siRNA molecule (or other nucleic acid) to a cell, e.g., a neuronal, fat, liver, or muscle cell. Several methods for expressing siRNA duplexes within cells from recombinant DNA constructs allow longer-term target gene suppression in cells, including mammalian Pol III promoter systems (e.g., HI or U6/snRNA promoter systems (Tuschl, Nature Biotechnol., 20:440-448, 2002) capable of expressing functional double-stranded siRNAs; (Bagella et al., J. Cell. Physiol, 177:206-1998; Lee et al., Nature Biotechnol., 20:500-505, 2002; Paul et al., Nature Biotechnol., 20:505-508, 2002; Yu et al., Proc. Natl. Acad. Sci. USA, 99(9):6047-6052, 2002; Sui et al., Proc. Natl. Acad. Sci. USA, 99(6):5515-5520, 2002). Transcriptional termination by RNA Pol III occurs at runs of four consecutive T residues in the DNA template, providing a mechanism to end the siRNA transcript at a specific sequence. The siRNA is complementary to the sequence of the target gene in 5 ‘-3’ and 3 ‘-5’ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998, supra; Lee et al., 2002, supra; Paul et al., 2002, supra; Yu et al., 2002, supra; Sui et al., 2002, supra). Constructs containing siRNA sequence under the control of T7 promoter also make functional siRNAs when cotransfected into the cells with a vector expression T7 RNA polymerase (Jacque, Nature, 418:435-438, 2002).

Animal cells express a range of noncoding RNAs of approximately 22 nucleotides termed micro RNA (miRNAs) and can regulate gene expression at the post transcriptional or translational level during animal development. miRNAs are excised from an approximately 70 nucleotide precursor RNA stem-loop. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng, Mol. Cell, 9:1327-1333, 2002). When expressed by DNA vectors containing polymerase III promoters, micro-RNA designed hairpins can silence gene expression (McManus, RNA 8:842-850, 2002). Viral-mediated delivery mechanisms can also be used to induce specific silencing of targeted genes through expression of siRNA, for example, by generating recombinant adenoviruses harboring siRNA under RNA Pol II promoter transcription control (Xia et al., Nat Biotechnol., 20(10): 1006-10, 2002).

Injection of the recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari et al, Proc. Natl. Acad. Sci. USA, 99: 14236-14240, 2002). In adult mice, efficient delivery of siRNA can be accomplished by “high-pressure” delivery technique, a rapid injection (within 5 seconds) of a large volume of siRNA containing solution into animal via the tail vein (Liu, Gene Ther., 6: 1258-1266, 1999; McCaffrey, Nature, 418:38-39, 2002; Lewis, Nature Genetics, 32:107-108, 2002). Nanoparticles and liposomes can also be used to deliver siRNA into test subjects. Likewise, in some embodiments, viral gene delivery, direct injection, nanoparticle particle-mediated injection, or liposome injection may be used to express siRNA in humans.

In some cases, a pool of siRNAs is used to modulate the expression of a target gene. The pool is composed of at least 2, 3, 4, 5, 8, or 10 different sequences targeted to the target gene. siRNAs or other compositions that inhibit target protein expression or activity are effective for ameliorating undesirable effects of a disorder related to TDP-43 mediated toxicity when target RNA levels are reduced by at least 25%, 50%, 75%, 90%, or 95%. In some cases, it is desired that target RNA levels be reduced by not more than 10%, 25%, 50%, or 75%. Methods of determining the level of target gene expression can be determined using methods known in the art. For example, the level of target RNA can be determined using Northern blot detection on a sample from a cell line or a subject. Levels of target protein can also be measured using, e.g., an immunoassay method.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following example is provided to illustrate certain embodiments of the invention. It is not intended to limit the invention in any way.

### Example I

Materials and methods are provided below to facilitate the practice of the present invention.

### Patient Selection.

63 patients (52 analyzable), with primary or recurrent operable GIST were enrolled onto the RTOG S0132 trial from 18 institutions. Patients' GIST samples were screened for CD 117 (KIT) positivity by standard IHC prior to participation in the clinical trial. Patients were required to have adequate hematologic, renal, and hepatic function as well as measurable disease for response evaluation. All patients signed informed consent following IRB approval for this study and were consented to provide baseline biopsies and operative tissue.

### Collection of Samples.

Tumor samples were obtained from pre-IM core needle biopsies (pre-treatment samples) and from the surgical specimen obtained at the time of resection following neoadjuvant/preoperative IM (post-treatment samples). A total of 48 pre- and 34 post-imatinib treated samples were collected and banked. All patients received IM at 600 mg daily by mouth which was continued daily until the day of surgery, with dose modifications for protocol defined toxicities. Fresh-frozen pre- and post-treatment GIST samples were collected from all participating institutions and shipped to the RTOG tissue bank prior to evaluation.

### RNA Isolation.

Total RNA was isolated from all available pre- and post-frozen tissue samples using TRIzol reagent according to the protocols provided by the manufacturer (Invitrogen Corp., Carlsbad, Calif.). RNA quantification and quality assessment were performed on 2100 Bioanalyser (Agilent Technologies, Santa Clara, Calif.). Due to the high variability in tissue collection and handling, storage and shipping procedures among the 18 institutions involved in the study and the tumor cellularity of the specimens, 35% (17 of 48) of pre- and 26% (9 of 34) of post-treatment samples were of limited quality and were therefore excluded from the gene profiling studies. Furthermore, one of the samples was excluded because the CT response data was lacking.

### DNA Isolation.

Genomic DNA was isolated as previously described (25). Quality DNA was isolated from 38 cases (2 pre-treatment biopsies and 36 post-treatment samples) and used for mutational analyses.

### KIT and PDGFRA Mutational Status Analysis.

Mutational analysis was performed as previously described (26).

### RNA Amplification and Microarray Hybridization.

Fifty nanograms (50 ng) of RNA from the various tissue samples, as well as 50 ng of Universal Human Reference RNA (Stratagene, La Jolla, Calif.) were amplified using Ovation Aminoallyl RNA amplification and labeling system (NuGEN Technologies, Inc., San Carlos, USA). Aminoallyl cDNA was purified with QIAquick PCR Purification Kit (Qiagen, Valencia, Calif.) and yield was measured using Spectrophotometer ND-1000 (NanoDrop, Wilmington, Del.). Sample aminoallyl cDNA was labeled with Alexa Fluor 647 dye (Invitrogen Corp) and reference aminoallyl cDNA was labeled with Alexa Fluor 555 dye (Invitrogen) as follows. Content of one vial from Alexa Fluor Reactive Dye Decapacks for Microarray Applications (Invitrogen) was resuspended in 2.5 μl of DMSO (Clontech, Mountain View, Calif.) and added to 2 mg of aminoallyl cDNA, which was previously dried down in vacuum centrifuge and resuspended in 7.5 μl of coupling buffer (66.5 mM NaHCO3, pH=9.0). After incubation for 1 hour in darkness at room temperature reaction was purified with QIAquick PCR Purification Kit (Qiagen). Labeling efficiency was assessed on Spectrophotometer ND-1000 (NanoDrop). Labeled sample and reference were combined and hybridized on 44K Whole Human Genome Oligo Microarray (Agilent) at 60° C. for 17 hours. Washing was performed in 6×SSPE buffer with 0.005% Sarcosine at room temperature for 1 min; 0.06×SSPE buffer with 0.005% Sarcosine at room temperature for 1 min, and then treated with Agilent Stabilization and Drying Solution at room temperature for 30 seconds.

### Data Analysis.

For the microarray studies we were able to obtain high quality RNA and array data from 28 pre-treatment samples and 25 post-treatment samples. For 17 we had matching pairs. Amplified and labeled RNAs were competitively hybridized against Stratagene Human Reference RNA using Agilent 4112a Whole Genome Human microarrays, scanned with an Agilent GMS 428 scanner, and preprocessed using the Functional Genomics Data Pipeline (27). These arrays were checked for quality by both Agilent quality control and by visual inspection of MA plots pre- and post-LOESS normalization (width=0.7, no background correction). Arrays that were of poor quality (i.e., which showed signs of RNA degradation such as splitting of MA plots into two ‘wings’) were repeated on a second RNA isolation from the same biopsy or tumor sample.

Clinical RECIST response is typically defined as a 30% decrease in the longest tumor diameter in the case of a primary target lesion or the sum of the longest diameters in the case of index tumors of metastatic disease. For the purpose of this analysis, as surgery occurred at a median of 65 days from the start of IM therapy, we arbitrarily divided these patients into Group A (≧25% tumor shrinkage after 8-12 weeks of IM) or Group B (<25% tumor shrinkage, unchanged, or evidence of tumor enlargement after 8-12 weeks of IM). In the seminal phase II metastatic GIST study the median time to partial response (PR) (≧30% reduction) was 16 weeks, therefore, we concluded that the duration of pre-op IM was probably too short to expect a significant number of patients having a classic PR per RECIST. We therefore chose an arbitrary grouping of CT measured response for patients in Group A of ≧25% close to the 30% RECIST criteria for PR. Had we selected ≧30% decreased in tumor dimension there would have been too few patients in Group A for any meaningful analysis. All other patient's gene array samples that correlated clinically to ≦25% decrease in tumor measurements as determined by the study clinical parameters were then placed in Group B. The 28 pre-treatment samples were analyzed with Significance Analysis of Microarrays (SAM) 28 implemented in the Multi-Experiment Viewer (MEV) 29 to identify genes that showed significant pretreatment differential expression between the two groups. A false discovery rate of 10% was used. Microarrays were annotated using the most recent (20 Aug. 2007) Agilent annotation file. The most current accession number corresponding to Agilent IDs were retrieved from the file. Ensembl accession numbers were annotated with gene symbols and descriptions on Jun. 6, 2008. Genebank accession numbers or gene names were annotated with NCBI Entrez information on Jun. 9, 2008.

Since 10 of the differentially expressed genes mapped to the same locus (HSA19p12-19p13.1), we also analyzed all of the genes in this locus for response upon treatment (25 post-treatment samples, with 13 samples from Group B and 12 from Group A) with IM. We performed this test by looking at each gene individually and looking for its average response in four categories: Group A pre-treatment, Group B pre-treatment, Group A post-treatment, Group B post-treatment. Microarray data including original Agilent scanner output files for all samples used in this study are available through the Gene Expression Omnibus (GEO).

### Quantitative RT-PCR.

To confirm the microarray data, RNA was freshly isolated from 9 of the trial's pre-IM samples (RTOG19, 22, 31, 39, 47, 56) including 3 samples (RTOG25, 35, and 53) not included in the original microarray analyses and reverse transcribed to cDNA by SuperScript II reverse transcriptase (Invitrogen). Expression of RNA for three KRAB-ZNF genes (ZNF 91, ZNF 43 and ZNF 208) and two endogenous control genes (HPRT and 18S) was measured in each pre-sample by real-time PCR (with TaqMan Gene Expression Assay products on an ABI PRISM 7900 HT Sequence Detection System, Applied Biosystems, Foster, Calif.) following protocols recommended by the manufacturer and as previously described 30. The relative mRNA expressions of ZNF 91, ZNF 43 and ZNF 208 were adjusted with either HPRT or actin. The primer/probe (FAM) sets for ZNF 91, ZNF 43, ZNF 208, HPRT and 18S were obtained from Applied Biosystems.

siRNA Transfection and IM Sensitivity.

Two siRNAs against each ZNF of interest (Qiagen) were pooled together and GIST cells were reverse transfected in four 96-well plates as described according to the protocols provided by the manufacturer (Qiagen). In addition, siRNA smart pools against KIT and GL-2 (Dharmacon) were used as positive and negative controls, respectively, and used for Z-score calculations. Forty-eight hours later vehicle only or vehicle+IM (45 nM) were added to two plates. After twenty-four hours cell viability was assessed using the cell titer blue assay. This assay is based on the ability of living cells to convert the redox dye, resazurin, into the fluorescent end product, resorufin. Cell titer blue was added to all wells and incubated for four hours followed by data recording using an EnVision microplate reader (PerkinElmer).

Representative siRNAs include the following:

Gene Symbol ZNF 43
(SEQ ID NO: 1)
CACATCAGGATAAAGTATCTA
Qiagen Cat. No. S104274592
Gene Symbol ZNF 43
(SEQ ID NO: 2)
TTGGTTGATAGTACAAAGTTT
Qiagen Cat. No. S103246012
Gene Symbol ZNF 91
(SEQ ID NO: 3)
TAGACAATCCTTAACCCTTAA
Qiagen Cat. No. S103226713
Gene Symbol ZNF 91
(SEQ ID NO: 4)
AAGCATTTATATCATCCTCAA
Qiagen Cat No. S100778988
Gene Symbol ZNF 85
(SEQ ID NO: 5)
TGGAACAAACTACAAGTGCAA
Qiagen Cat No. S103119277
Gene Symbol ZNF 208
(SEQ ID NO: 6)
AAAGCCCTGGATCATATGAAA
Qiagen Cat. No. S104245815
Gene Symbol ZNF 208
(SEQ ID NO: 7)
CTGGTTGTCAGTCTTTAGTAA
Qiagen Cat No. S10069909
Gene Symbol GTF2I
(SEQ ID NO: 8)
TAGGTGGTCGTGTGATGGTAA
Gene Symbol RASSF8
(SEQ ID NO: 9)
CCGGTGCACCATGGAACTTAA
Gene Symbol ZNF100
(SEQ ID NO: 10)
CCTGCTAAAGTTAGCTTGTAA
Gene Symbol ZNF254
(SEQ ID NO: 11)
TTCGACAATGCTCACACCCTA
Gene Symbol ZNF429
(SEQ ID NO: 12)
CTCAACACTTACTCAAGACAA
Gene Symbol ZNF431
(SEQ ID NO: 13)
CACGCCCAGCCTGTAGCATAT
Gene Symbol ZNF528
(SEQ ID NO: 14)
CAGACCTTATACGACATCGAA
Gene Symbol ZNF665
(SEQ ID NO: 15)
AAACACGGATTTGCCACCAAA
Gene Symbol ZNF708
(SEQ ID NO: 16)
CTGGCTCTTAATCCTTATGAA
Gene Symbol IGF2R
(SEQ ID NO: 17)
CAGAGATTACCTGGAAAGTAA
Gene Symbol IGFBP2
(SEQ ID NO: 18)
CACACGTATTTATATTTGGAA

### Results

### RTOG-S0132 Trial Design and Patient Response to IM

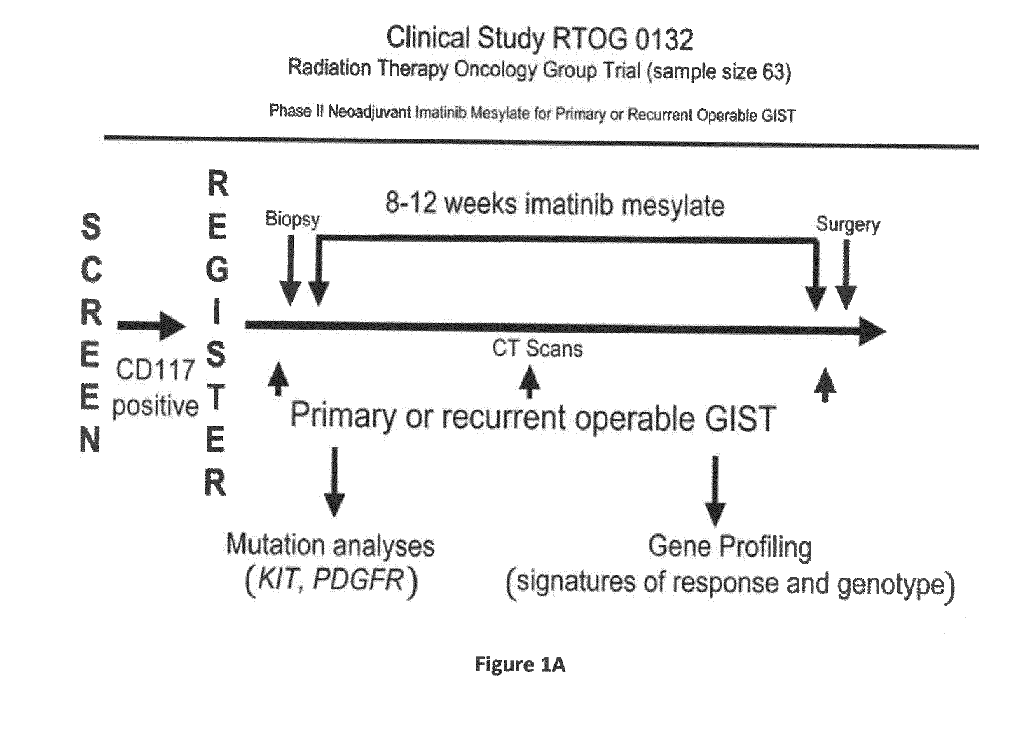

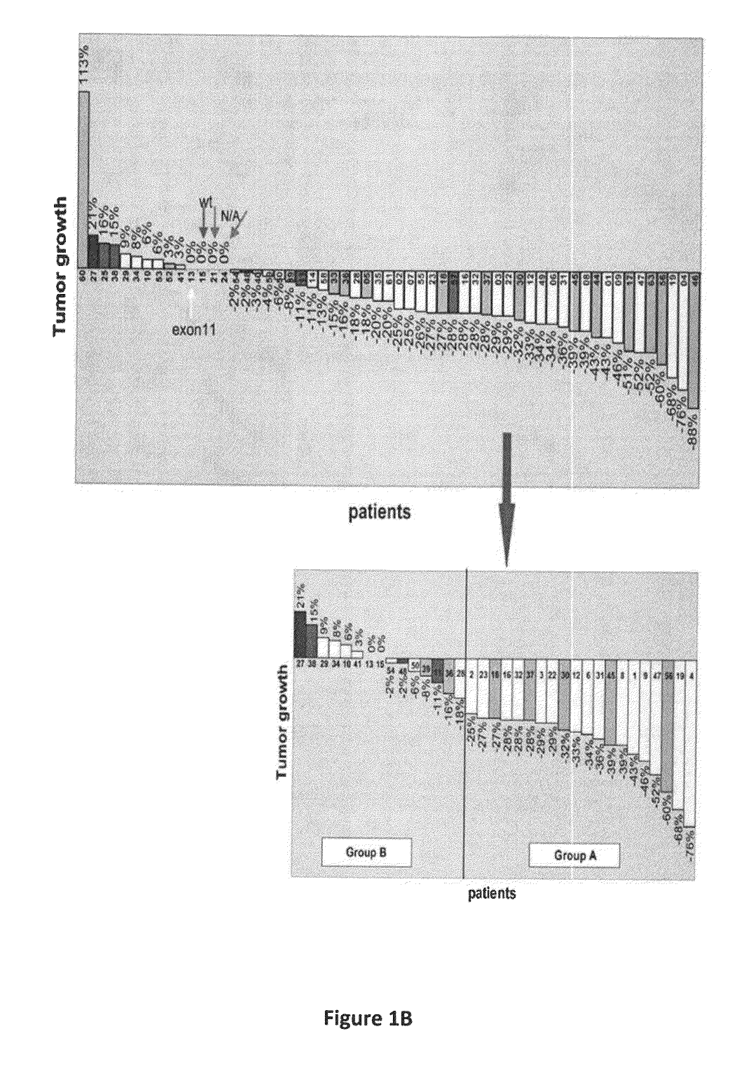

Sixty-three (63) patients with primary or recurrent potentially resectable malignant GIST, from 18 institutions, were originally enrolled onto the trial beginning in February 2002 and ending in June 2006 (15). A tumor positive for KIT (CD117) staining by IHC was the necessary prerequisite for patient enrollment. Fifty-three percent (53%) of primary tumors were located in the stomach, 27% in small bowel, and 20% in GI other sites. Metastatic tumors were primarily located in the abdomen/peritoneum. Additional clinical information is shown in Table 1. Prior to the start of the 8-12 week IM regimen, a CT scan was performed and a tumor biopsy (pre-treatment sample) was obtained. CT scans were repeated ˜4-6 weeks into IM therapy and again immediately prior to surgical resection (after 8-12 weeks IM therapy) (FIG. 1A). CT measurements, taken from the longest cross sectional diameter of the primary GIST or the index metastatic lesion(s), were used to assess tumor response (i.e. tumor shrinkage, no measurable change, or tumor enlargement) to IM therapy (FIG. 1B). Of the 52 analyzable patients, 58% (30 of 52) had surgical resection of primary locally advanced GIST, whereas 42% (22 of 52) had recurrent/metastatic GIST resected. Genomic DNA was isolated from available large biopsies (pre-treatment samples) or resected tumor (post-treatment samples) and KIT and PDGFRA mutational analysis was performed (FIG. 1B). Mutational analysis was performed on 39 of the 52 patients and the most frequent mutations occurred in exon 11 (82%, 32 of 39), followed by exon 9 (3%, 1/39). No mutations were found in exons 13 and 17 of KIT or in exons 12, 14 and 18 of PDGFRA. Fifteen percent (15%, 6 of 39) of 15 the patients tested lacked mutations in both KIT and PDGFRA. Similar frequencies have been observed previously (12).

TABLE 1
Patients' and tumors' characteristics.

| | n (%) | |
|---|---|---|
| Median age (range), years | 58.5 | (24 to 84) |
| Sex | | |
| Female | 24 | (46) |
| Male | 28 | (54) |
| Primary tumor | 30 | (58) |
| Metastatic/recurrent tumor | 22 | (42) |
| Site of primary tumor | | |
| Stomach | 16 | (53) |
| Small bowel | 8 | (27) |
| Other | 6 | (20) |
| Site of metastatic tumor | | |
| Abdomen/peritoneum | 15 | (68) |
| Liver only | 6 | (27) |
| Liver/peritoneum | 1 | (5) |
| Size of tumor (cm) | | |
| ≦10 | 37 | (71) |
| >10 | 15 | (29) |
| Mutation | | |
| Exon 11 KIT | 32 | (62) |
| Exon 9 KIT | 1 | (2) |
| Exon 17 KIT | 0 | (0) |
| PDGFRα (exons 18 and 12) | 0 | (0) |
| Wild-type | 6 | (12) |
| N/A* | 13 | (25) |

*Not enough tissue for mutational analysis.

Gene expression profiles associated with response to IM.

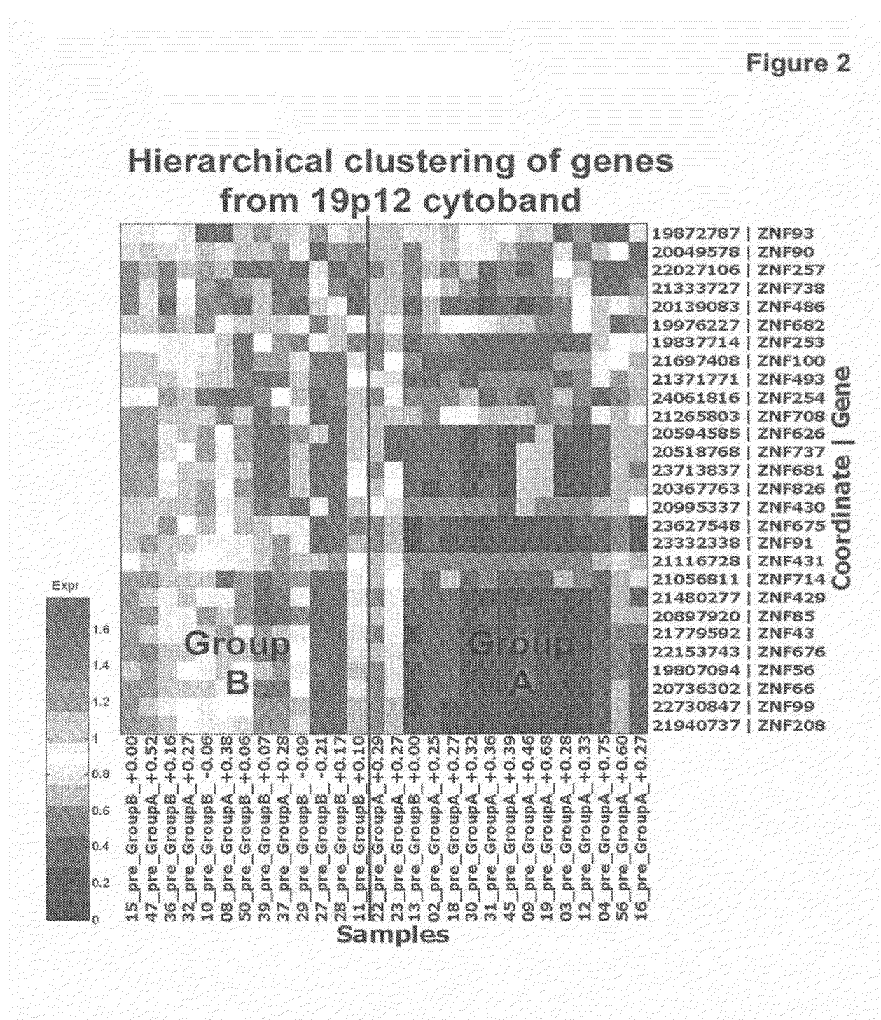

RNA was isolated from both pre- and post-treatment samples and those deemed of adequate amount and quality were evaluated by using Agilent oligonucleotide microarrays (see Methods). GIST specimens (pre-, post- or both) used for microarray analysis are shown in FIG. 1B (bottom). CT measurements were used to classify patients as either “immediate responders” (Group A) if the patient's tumor demonstrated a 25% or greater reduction in size during the 8 to 12 weeks of IM treatment. The other GIST samples were combined and will subsequently be referred to as Group B. The index used for these latter tumors ranged from an 18% diameter reduction to a 21% tumor enlargement after 8-12 weeks of IM. The SAM analysis identified 38 genes as differentially expressed at a false discovery rate of 10% in pre-treatment samples between the two groups, with all gene transcripts present at higher levels in patients within Group B (Table 2). Thirty-two (32) of these corresponded to known genes, 18 of these are Krüppel-associated box (KRAB)-zinc finger (ZNF) genes, 10 of which mapped to the same locus (HSA19p12-19p13.1), and 2 have similarity to ZNF 91 and ZNF 208 (FIG. 2). Some of the remaining genes within this signature encode for the zinc finger-containing proteins (ZMYND11 and ZMAT1) and transcription factors, such as GTF2I and GABPAP. Two additional genes in the signature are ZNF genes that map to 19q13.41.

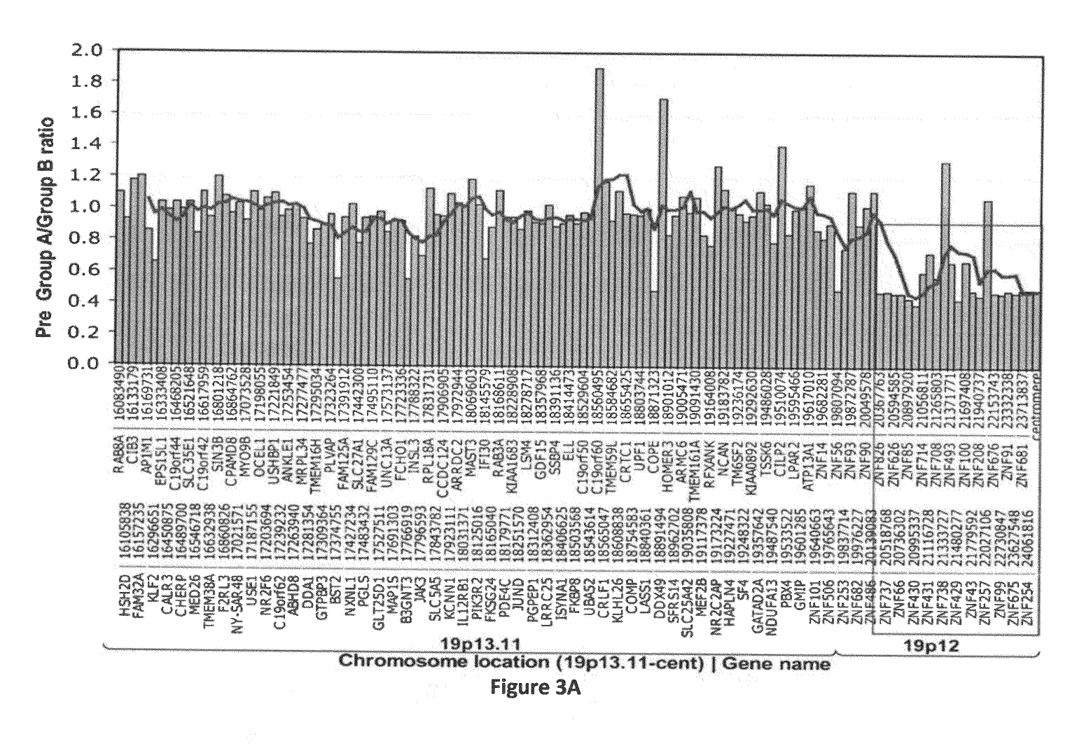

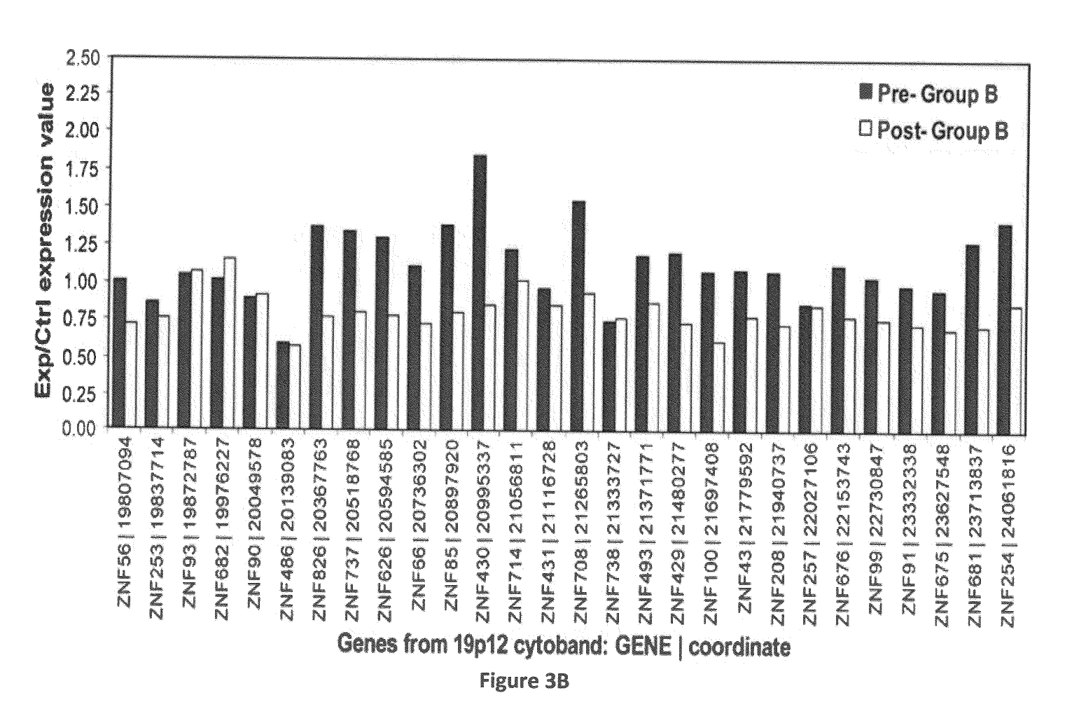

Analysis of pre-treatment sample expression differences for all genes within the 19p12-13.1 locus showed a consistent difference (FIG. 3A, red box). All the ZNF genes showed higher overall expression in samples from patients within Group B across the locus, even though adjoining genes showed equal expression between the two groups. Of additional interest, these KRAB-ZNFs appear to be coordinately regulated in response to IM therapy in that KRAB-ZNF mRNA levels decrease in tumors from patients in Group B after IM. In order to rule out the possibility that an enrichment of other non-tumor cells, such as endothelial and inflammatory cells may be contributing to the observed expression patterns we examined the cellular content of the post-IM samples and used only those that displayed >70% tumor cellularity (FIG. 3B). We also observed a very similar pattern of decreased ZNF expression in the Group B post-IM samples with lower tumor (<70%) cellularity (data not shown), suggesting that the observed trend is likely associated with tumor cell response to IM. Analysis of the pre- and post-treatment samples from Group A showed an opposing trend in that the level of ZNF genes increased following the 8-12 week IM regimen; however, since the cellularity was <70% for all but one of these samples we cannot rule out the effect of non-tumor cells on these expression patterns (data not shown).

TABLE 2
SAM (significance analysis of microarrays) analysis of genes differentially
expressed between rapid responders and stable disease.

| | Gene | | | SAM |
|---|---|---|---|---|
| Accession | symbol | Description | Cytoband | score |
| NM_178549 | ZNF678 | zinc finger protein 678 | 1q42.13 | 4.10 |
| NM_212479 | ZMYND11 | zinc finger, MYND domain containing 11 | 10p15.3 | 4.11 |
| NM_007211 | RASSF8 | Ras association (RalGDS/AF-6) domain family 8 | 12p12.1 | 3.55 |
| A_24_P75888 | n/a | n/a | 14q11.1 | 4.35 |
| AK126622 | WDR90 | WD repeat domain 90 | 16p13.3 | 3.61 |
| A_24_P717262 | n/a | n/a | 19p12 | 4.23 |
| ENST00000341262 | ZNF56 | zinc finger protein 56 (Fragment) | 19p12 | 3.97 |
| AK131420 | ZNF66 | zinc finger protein 66 | 19p12 | 4.06 |
| NM_003429 | ZNF85 | zinc finger protein 85 | 19p12 | 4.36 |
| NM_133473 | ZNF431 | zinc finger protein 431 | 19p12 | 3.90 |
| NM_001001415 | ZNF429 | zinc finger protein 429 | 19p12 | 4.29 |
| NM_003423 | ZNF43 | zinc finger protein 43 | 19p12 | 4.10 |
| NM_007153 | ZNF208 | zinc finger protein 208 | 19p12 | 3.69 |
| NM_001001411 | ZNF676 | zinc finger protein 676 | 19p12 | 4.08 |
| ENST00000357491 | LOC646825 | DISCONTINUED: similar to zinc finger protein 91 | 19p12 | 4.14 |
| NM_001080409 | ZNF99 | zinc finger protein 99 | 19p12 | 3.84 |
| XR_017338 | LOC388523 | similar to zinc finger protein 208 | 19p12 | 4.10 |
| NM_003430 | ZNF91 | zinc finger protein 91 | 19p12 | 3.95 |
| ENST00000334564 | ZNF528 | zinc finger protein 528 | 19q13.33 | 3.92 |
| NM_024733 | ZNF665 | zinc finger protein 665 | 19q13.41 | 3.74 |
| NM_001004301 | ZNF813 | zinc finger protein 813 | 19q13.41 | 3.86 |
| AK001808 | n/a | CDNA FLJ10948 fis, clone PLACE1000005 | 2q24.3 | 4.21 |
| BE168511 | SF3B1 | Splicing factor 3b, subunit 1, 155 kDa | 2q33.1 | 3.86 |
| NM_138402 | LOC93349 | hypothetical protein BC004921 | 2q37.1 | 4.42 |
| ENST00000305570 | LOC727867 | similar to PRED65 | 21q11.2 | 3.57 |
| ENST00000341087 | n/a | n/a | 4p16.3 | 4.53 |
| NM_001074 | UGT2B7 | UDP glucuronosyltransferase 2 family, polypeptide B7 | 4q13.2 | 3.66 |
| NM_182524 | ZNF595 | zinc finger protein 595 | 4p16.3 | 3.71 |
| THC2708803 | n/a | n/a | 4q22.3 | 3.85 |
| A_24_P492885 | n/a | n/a | 7q11.21 | 4.39 |
| XM_001127354 | LOC728376 | similar to hCG1996858 | 7p11.2 | 4.48 |
| AF277624 | ZNF479 | zinc finger protein 479 | 7p11.2 | 4.19 |
| NR_002723 | GABPAP | GA binding protein TF, alpha subunit pseudogene | 7q11.21 | 4.14 |
| XM_001128828 | LOC728927 | similar to hCG40110 | 7q11.21 | 4.05 |
| NM_178558 | ZNF680 | zinc finger protein 680 | 7q11.21 | 3.59 |
| NM_001518 | GTF2I | general transcription factor II, I | 7q11.23 | 4.09 |
| NM_197977 | ZNF189 | zinc finger protein 189 | 9q31.1 | 3.64 |
| NM_032441 | ZMAT1 | zinc finger, matrin type 1 | Xq22.1 | 3.74 |

### Validation with qRT-PCR

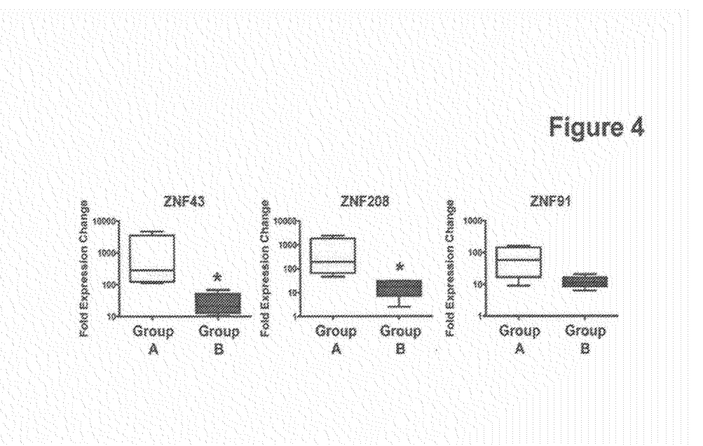

We used qRT-PCR to validate the differential expression pattern of the predictor genes. For this analysis, four genes were selected from the list of 18 KRAB-ZNF genes identified in the microarray analysis based on availability of commercial qRT-PCR assays. We found the assays for ZNF 43, ZNF 208 and ZNF 91 to work reliably. All three were expressed significantly higher in Group B prior to IM treatment compared to the immediate response group. The expression of each gene was evaluated in a small validation panel consisting of nine pre-treatment samples from patients on the trial for which high quality RNAs could be isolated (see Methods). ZNF 43, ZNF 208, and ZNF 91 mRNA levels were significantly lower in patients whose tumors rapidly shrunk in response to IM than in those who did not (FIG. 4). Expression levels of the three genes were highly correlated with each other (all pairwise correlations were greater than 0.93 with p values <0.0003).

We next sought to determine if modifying the expression of a subset of the genes within this predictive signature could alter the sensitivity of GIST cells to IM. We selected ZNF 208, ZNF 91, ZNF 85 and ZNF 43 for siRNA targeted knockdown. From these screens, we demonstrated that depletion of each of the four ZNFs were able to sensitize GIST cells to varying degrees of IM (Sensitization Index=viability with drug/viability with vehicle only was 0.58 to 0.85). These findings suggest that some members of this gene signature may not only have predictive value but functional relevance to IM activity in vivo. We also developed genomic-based qPCR analysis to assess gene copy number of these KRAB-ZNF genes. We found that upregulation of these ZNFs in patients within Group B was not associated with gene amplification (data not shown), indicating that the changes in mRNA were independent of gene copy number.

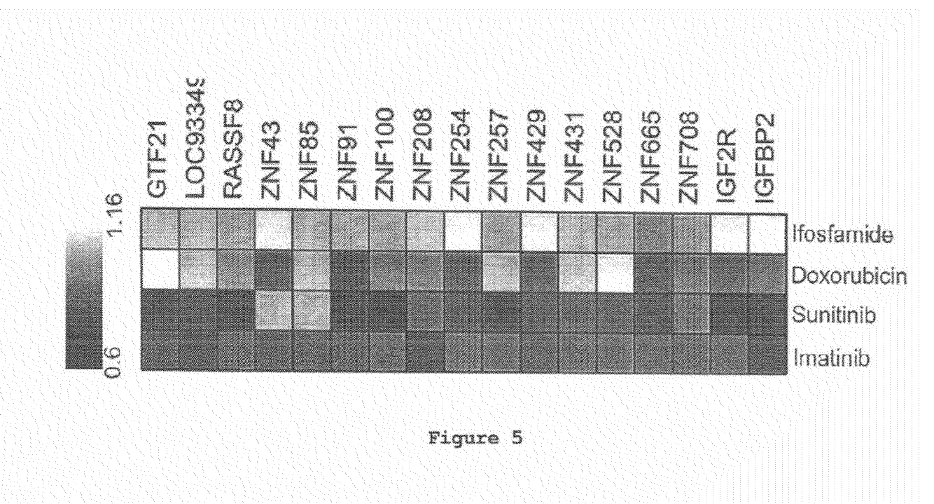

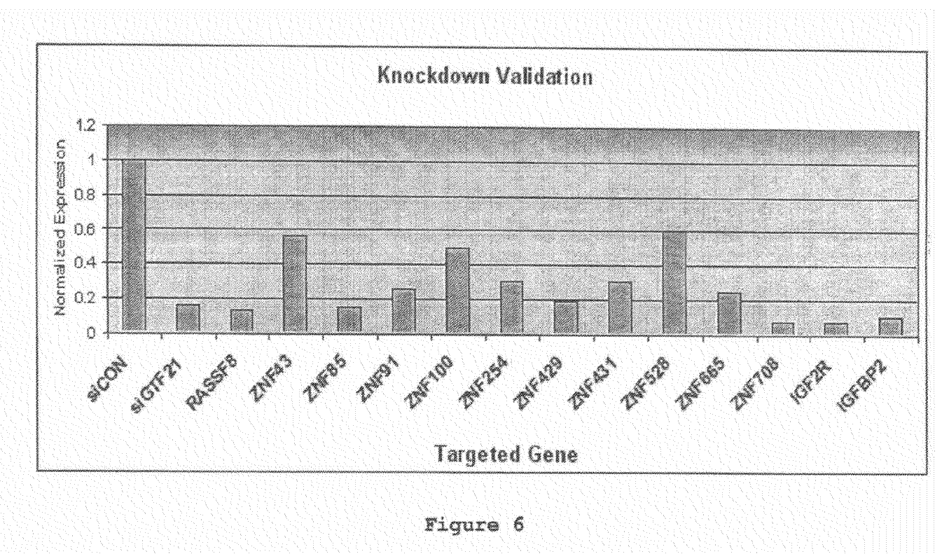

The gene signature described above is predictive of likely rapid response to short-term IM treatment and thus provides a prognostic biomarker for identifying those patients who will benefit from such treatment. This gene signature is composed of thirty-eight genes that were expressed at significantly lower levels in the pre-treatment samples of tumors that rapidly responded to IM. Eighteen of these genes encoded KRAB domain containing zinc finger (KRAB-ZNF) transcriptional repressors, and importantly, ten mapped to a single locus on chromosome 19p. In further experiments for determining if modifying expression of genes within this predictive signature were functionally associated with response to IM and could enhance the sensitivity of GIST cells to this drug, we designed a custom siRNA library targeting all the genes within the predictive signature. From these screens we have identified 17 genes as “IM sensitizing hits”<0.85 ratio of drug/vehicle) with a false discovery rate (FDR) <5% (FIG. 5, bottom row). These 17 hits were validated by confirming that 2 or more (of 4) independent siRNAs targeting the same gene in each case provided sensitization to IM <0.85 ratio of drug/vehicle, and FDR <5%). Interestingly, 12 of the 17 (71%) validated hits were the ZNF genes, 10 (59%) of which (59%) are located within the 19p12-13.1 locus. Quantitative PCR analysis confirmed knockdown of the target in 14/17 validated genes (FIG. 6). In addition, the validation set has been tested with other agents to measure IM specificity. We selected doxorubicin (adriamycin) (FIG. 5, second row) and ifosfamide (FIG. 5, top row), chemotherapeutic agents used prior to IM to treat GISTs that for the most part were ineffective therapies, as well as sunitinib (FIG. 5, third row), a small molecule kinase inhibitor which has shown some success in the treatment of GISTs. None of these genes were sensitizing hits for ifosfamide and knockdown of 5 out of 17 (29%) of these genes sensitized GIST T1 cells to doxorubicin, whereas knockdown of 14 out of 17 genes (82%) sensitized to sunitinib. These findings are significant given that sunitinib has many of the same down-stream signaling targets as IM. Overall, we have identified a gene signature that includes KRAB-ZNF 91 subfamily members that is both predictive of and functionally associated with likely response to short term IM treatment.

### DISCUSSION

In this study, we set out to obtain a gene expression profile that could be predictive of likely IM induced cytoreduction in GIST patients prior to therapy. Because several alternative options for progressive disease treatment are currently being evaluated, such as new kinase inhibitors or combination therapy with IM, such a profile may be useful in determining appropriate personalized clinical treatment of GIST patients.

The clinical trial from which tissue samples were obtained for this study has yielded some interesting findings. The majority of patients on this trial had apparent clinical benefit from IM therapy prior to surgery. Forty-nine percent (49%) of all patients enrolled onto the trial manifested ≧25% tumor size reduction following the initiation of 8-12 weeks of IM therapy, with 75.4% having at least some degree of tumor response (FIG. 1B). In addition, pre-operative IM therapy was associated with minimal drug related toxicity and surgical morbidity 31. We observed benefit from the neoadjuvant use of IM for downsizing tumors prior to surgical resection. Using pre-IM samples from this study we were able to perform microarray analysis to obtain a gene expression profile that may be indicative of the likely response to short-term IM therapy. Although expression of several interesting genes, such as RASSF8, SF3B1, and UGT2B7 were found to be associated with differential response to IM, we were drawn to the observation that nearly a third of the genes clustered in one locus on chromosome 19p12 near the centromere (FIG. 2). These differentially expressed ZNFs are KRAB-ZNF genes that are members of the ZNF 91 subfamily 32, 33. In addition, we demonstrated that expression of these ZNFs appeared to be coordinately regulated by IM treatment (FIG. 3B and data not shown).

The ZNF 91 subfamily includes 64 genes, 37 of which are found on chromosome 19 (32). These KRAB-ZNF proteins are characterized by the presence of a DNA-binding domain composed of between 4 and 30 zinc-finger motifs and a KRAB domain near the amino terminus. They form one of the largest families of transcriptional regulators. Many members of this family are still uncharacterized and the specific functions of many members are unknown; however, some of these ZNFs have been associated with undifferentiated cells and also implicated in cancers. Lovering and Trowsdale showed that expression of ZNF 43 was increased in lymphoid cell lines and that inducing terminal differentiation in vitro in one of these cell lines led to reduced ZNF 43 expression (34). Another study using microarrays comparing normal controls to mononuclear cells of AML patients, showed ZNF 91 expression was increased in 93% of AML cases and that inhibiting expression of ZNF 91 induced apoptosis of these cells (35). Eight other ZNFs, not found to reach significance in our tests for differential expression in our studies, have been denoted as “candidate cancer genes” or CAN-genes by largescale mutagenesis screens in breast and colorectal cancers (36).

In addition, KRAB-ZNF expression has been associated with resistance to IM. Using DNA microarrays, Chung et al. (2006) showed that 22 genes, 2 of which are ZNFs, were positively correlated with increasing IM dosage in chronic myelogenous leukemia cell lines (37). However, our study is the first to establish this connection in GIST patients and to the genes within the HSA19p12-19p13.1 locus. The ultimate goal of this work was to identify a profile that is indicative of immediate response to IM so that in the future, expression of these ZNFs can be examined in patient biopsies prior to treatment, allowing for the most effective therapeutic regimen to be employed, particularly in relation to planned surgical resection. Our study suggests, since there is a significant overexpression of these KRABZNFs in patients who are not as responsive to IM, that IHC or qRT-PCR expression analyses of these genes could potentially serve as a rapid means for pre-screening GIST patients prior to treatment. We have shown that qRT-PCR assays are informative when adequate RNA samples can be obtained either from small needle biopsies or resected tumor samples. Our studies also highlight the need for additional studies to assess the role of these KRAB-ZNFs in potentially mediating IM-response. In preliminary studies we have found that siRNA mediated targeted knockdown of ZNF 208, ZNF 91, ZNF 85 and ZNF 43 can enhance the sensitivity of GIST cells to IM, albeit to varying degrees. Further functional studies are currently underway to determine how these genes may be influencing IM activity in GISTs.

We also searched for links as to why many of these ZNF genes within a single locus are coordinately regulated at the expression level. Using transcription factor binding site analysis, from advanced biomedical computing center and viewed using CIMminer software, we sought to identify common transcription factors (TFs) that could explain why, in some samples, all the genes are either upregulated or downregulated. The analysis showed that there are a number of TFs that regulate these ZNFs (data not shown). One TF, HinfA, appeared to be associated with 12 of the ZNFs of interest. HinfA is a TF known to bind to A/T rich repeats in the promoters of human histone (H3 and H4) genes (38). However, HinfA was not measured on our array. Vogel and colleagues have found that the heterochromatin binding proteins, CBX1 and SUV39H1 have been associated with co-expression of ZNF genes (39). However, our analysis of the three probes for CBX1 and one probe for SUV39H1 did not detect significant differences in expression between these two groups.

In summary, we were able to elucidate a gene expression profile that is unique to patients whose tumors are less responsive to IM in comparison to those that rapidly respond. This profile consists of 32 genes, 18 of which are KRAB-ZNFs. We feel that these results have potential clinical relevance and could help stratify patients most responsive to IM, and potentially design more effective treatment regimens particularly in neoadjuvant use for GIST patients in the future.

### REFERENCES

1. Corless C L, Heinrich M C. Molecular pathobiology of gastrointestinal stromalsarcomas. Annual review of pathology 2008; 3:557-86.
2. El-Rifai W, Sarlomo-Rikala M, Andersson L C, Knuutila S, Miettinen M. DNA sequence copy number changes in gastrointestinal stromal tumors: tumor progression and prognostic significance. Cancer research 2000; 60:3899-903.
3. Miettinen M, Lasota J. Gastrointestinal stromal tumors: review on morphology, molecular pathology, prognosis, and differential diagnosis. Archives of pathology & laboratory medicine 2006; 130:1466-78.
4. Tworek J A, Appelman H D, Singleton T P, Greenson J K. Stromal tumors of the jejunum and ileum. Mod Pathol 1997; 10:200-9.
5. Miettinen M, Kopczynski J, Makhlouf H R, et al. Gastrointestinal stromal tumors, intramural leiomyomas, and leiomyosarcomas in the duodenum: a clinicopathologic, immunohistochemical, and molecular genetic study of 167 cases. The American journal of surgical pathology 2003; 27:625-41.
6. Tworek J A, Goldblum J R, Weiss S W, Greenson J K, Appelman H D. Stromal tumors of the anorectum: a clinicopathologic study of 22 cases. The American journal of surgical pathology 1999; 23:946-54.
7. Miettinen M, Monihan J M, Sarlomo-Rikala M, et al. Gastrointestinal stromal tumors/smooth muscle tumors (GISTs) primary in the omentum and mesentery: clinicopathologic and immunohistochemical study of 26 cases. The American journal of surgical pathology 1999; 23:1109-18.
8. Corless C L, Fletcher J A, Heinrich M C. Biology of gastrointestinal stromal tumors. J Clin Oncol 2004; 22:3813-25.
9. Mazur M T, Clark H B. Gastric stromal tumors. Reappraisal of histogenesis. The American journal of surgical pathology 1983; 7:507-19.
10. Graadt van Roggen J F, van Velthuysen M L, Hogendoorn P C. The histopathological differential diagnosis of gastrointestinal stromal tumours. Journal of clinical pathology 2001; 54:96-102.
11. Heinrich M C, Corless C L. Gastric GI stromal tumors (GISTs): the role of surgery in the era of targeted therapy. J Surg Oncol 2005; 90:195-207; discussion
12. Subramanian S, West R B, Corless C L, et al. Gastrointestinal stromal tumors (GISTs) with KIT and PDGFRA mutations have distinct gene expression profiles. Oncogene 2004; 23:7780-90.
13. Tarn C, Merkel E, Canutescu A A, et al. Analysis of KIT mutations in sporadic and familial gastrointestinal stromal tumors: therapeutic implications through protein modeling. Clin Cancer Res 2005; 11:3668-77.
14. Debiec-Rychter M, Sciot R, Le Cesne A, et al. KIT mutations and dose selection for imatinib in patients with advanced gastrointestinal stromal tumours. Eur J Cancer 2006; 42:1093-103.
15. Eisenberg B L, Judson I. Surgery and imatinib in the management of GIST: emerging approaches to adjuvant and neoadjuvant therapy. Annals of surgical oncology 2004; 11:465-75.
16. DeMatteo R P, Lewis J J, Leung D, Mudan S S, Woodruff J M, Brennan M F. Two hundred gastrointestinal stromal tumors: recurrence patterns and prognostic factors for survival. Annals of surgery 2000; 231:51-8.
17. Demetri G D, von Mehren M, Blanke C D, et al. Efficacy and safety of imatinib mesylate in advanced gastrointestinal stromal tumors. N Engl J Med 2002; 347:472-80.
18. Verweij J, Casali P G, Zalcberg J, et al. Progression-free survival in gastrointestinal stromal tumours with high-dose imatinib: randomised trial. Lancet 2004; 364:1127-34.
19. Van den Abbeele A D, Badawi R D. Use of positron emission tomography in oncology and its potential role to assess response to imatinib mesylate therapy in gastrointestinal stromal tumors (GISTs). Eur J Cancer 2002; 38 Suppl 5:S60-5.
20. Zalcberg J R, Verweij J, Casali P G, et al. Outcome of patients with advanced gastrointestinal stromal tumours crossing over to a daily imatinib dose of 800 mg after progression on 400 mg. Eur J Cancer 2005; 41:1751-7.
21. Prenen H, Cools J, Mentens N, et al. Efficacy of the kinase inhibitor SU11248 against gastrointestinal stromal tumor mutants refractory to imatinib mesylate. Clin Cancer Res 2006; 12:2622-7.
22. Weisberg E, Wright R D, Jiang J, et al. Effects of PKC412, nilotinib, and imatinib against GIST-associated PDGFRA mutants with differential imatinib sensitivity. Gastroenterology 2006; 131:1734-42.
23. Schittenhelm M M, Shiraga S, Schroeder A, et al. Dasatinib (BMS-354825), a dual SRC/ABL kinase inhibitor, inhibits the kinase activity of wild-type, juxtamembrane, and activation loop mutant KIT isoforms associated with human malignancies. Cancer research 2006; 66:473-81.
24. Bauer S, Yu L K, Demetri G D, Fletcher J A. Heat shock protein 90 inhibition in imatinib-resistant gastrointestinal stromal tumor. Cancer research 2006; 66:9153-61.
25. Godwin A K, Vanderveer L, Schultz D C, et al. A common region of deletion on chromosome 17q in both sporadic and familial epithelial ovarian tumors distal to BRCA1. American journal of human genetics 1994; 55:666-77.
26. Corless C L, McGreevey L, Haley A, Town A, Heinrich M C. KIT mutations are common in incidental gastrointestinal stromal tumors one centimeter or less in size. The American journal of pathology 2002; 160:1567-72.
27. Grant J D, Somers L A, Zhang Y, Manion F J, Bidaut G, Ochs M F. FGDP: functional genomics data pipeline for automated, multiple microarray data analyses. Bioinformatics (Oxford, England) 2004; 20:282-3.
28. Tusher V G, Tibshirani R, Chu G. Significance analysis of microarrays applied to the ionizing radiation response. Proceedings of the National Academy of Sciences of the United States of America 2001; 98:5116-21.
29. Saeed A I, Sharov V, White J, et al. TM4: a free, open-source system for microarray data management and analysis. BioTechniques 2003; 34:374-8.
30. Chen X, Arciero C A, Wang C, Broccoli D, Godwin A K. BRCC36 is essential for ionizing radiation-induced BRCA1 phosphorylation and nuclear foci formation. Cancer research 2006; 66:5039-46.
31. Heinrich M C, Corless C L, Blanke C D, et al. Molecular correlates of imatinib resistance in gastrointestinal stromal tumors. J Clin Oncol 2006; 24:4764-74.
32. Hamilton A T, Huntley S, Tran-Gyamfi M, Baggott D M, Gordon L, Stubbs L. Evolutionary expansion and divergence in the ZNF91 subfamily of primate-specific zinc finger genes. Genome research 2006; 16:584-94.
33. Mark C, Abrink M, Hellman L. Comparative analysis of KRAB zinc finger proteins in rodents and man: evidence for several evolutionarily distinct subfamilies of KRAB zinc finger genes. DNA and cell biology 1999; 18:381-96.
34. Lovering R, Trowsdale J. A gene encoding 22 highly related zinc fingers is expressed in lymphoid cell lines. Nucleic acids research 1991; 19:2921-8.
35. Unoki M, Okutsu J, Nakamura Y. Identification of a novel human gene, ZFP91, involved in acute myelogenous leukemia. International journal of oncology 2003; 22:1217-23.
36. Sjoblom T, Jones S, Wood L D, et al. The consensus coding sequences of human breast and colorectal cancers. Science (New York, N.Y. 2006; 314:268-74.
37. Chung Y J, Kim T M, Kim D W, et al. Gene expression signatures associated with the resistance to imatinib. Leukemia 2006; 20:1542-50.
38. van Wijnen A J, Massung R F, Stein J L, Stein G S. Human H1 histone gene promoter CCAAT box binding protein HiNF-B is a mosaic factor. Biochemistry 1988; 27:6534-41.
39. Vogel M J, Guelen L, de Wit E, et al. Human heterochromatin proteins form large domains containing KRAB-ZNF genes. Genome research 2006; 16:1493-504.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1-24. (canceled)

25. A kit comprising a Gene Chip comprising isolated differentially expressed sequence markers having accession numbers selected from the group consisting of

| | |
|---|---|
| NM_178549 | ZNF678 |
| NM_212479 | ZMYND11 |
| NM_007211 | RASSF8 |
| AK126622 | WDR90 |
| ENST00000341262 | ZNF56 |
| AK131420 | ZNF66 |
| NM_003429 | ZNF85 |
| NM_133473 | ZNF431 |
| NM_001001415 | ZNF429 |
| NM_003423 | ZNF43 |
| NM_007153 | ZNF208 |
| NM_001001411 | ZNF676 |
| ENST00000357491 | LOC646825 |
| NM_001080409 | ZNF99 |
| XR_017338 | LOC388523 |
| NM_003430 | ZNF91 |
| ENST00000334564 | ZNF528 |
| NM_024733 | ZNF665 |
| NM_001004301 | ZNF813 |
| BE168511 | SF3B1 |
| NM_138402 | LOC93349 |
| ENST00000305570 | LOC727867 |
| NM_001074 | UGT2B7 |
| NM_182524 | ZNF595 |
| XM_001127354 | LOC728376 |
| AF277624 | ZNF479 |
| NR_002723 | GABPAP |
| XM_001128828 | LOC728927 |
| NM_178558 | ZNF680 |
| NM_001518 | GTF2I |
| NM_197977 | ZNF189 |
| NM_032441 | ZMAT1, |

for identifying patients likely to benefit from treatment of GIST with imatinib mesylate (IM), said kit also comprising a listing of expression of levels of said markers associated with an increased risk of resistance, a container, and optionally, means for obtaining a biopsy or cell sample.

26. The kit of claim 25 comprising reagents for amplifying said differentially expressed sequence markers present in said sample which are indicative of an increased risk of resistance to IM therapy.

27. A method of identifying patients likely to benefit from treatment of GIST with imatinib mesylate (IM), comprising,
a) providing a genetic signature comprising differentially expressed nucleic acids obtained from cells which are sensitive to IM in a kit according to claim 1;
b) obtaining nucleic acids from cells isolated from said patient having GIST which correspond to the differentially expressed nucleic acids of step a; and
c) assessing expression levels of at least one of said differentially expressed nucleic acids, wherein an increase in the expression level of said at least one gene product in said patient relative to expression levels observed in cells responsive to IM, is indicative of an increased risk of resistance to IM therapy.

28. The method of claim 27, wherein expression levels of at least one nucleic acid selected from the group consisting of ZNF 208, ZNF 91, ZNF 85, ZNF 43, GTF2I, LOC93349, RASSF8, ZNF100, ZNF254, ZNF429, ZNF431, ZNF528, ZNF665, ZNF708, IGF2R, and IGFBP2 is determined in said patient sample, increased expression levels in said patient sample relative to those observed in the step a) being indicative of an increased risk of resistance to IM therapy.

29. The method of claim 28, wherein expression levels of five genes showing the greatest amount of differential expression are assessed.

30. The method of claim 27, wherein said patient has refractory GIST.


SEQUENCE LISTING (18 sequences)

SEQ ID NO: 1 - DNA, 21 bp, Artificial Sequence
  siRNA
cacatcaggataaagtatcta

SEQ ID NO: 2 - DNA, 21 bp, Artificial Sequence
  siRNA
ttggttgatagtacaaagttt

SEQ ID NO: 3 - DNA, 21 bp, Artificial Sequence
  siRNA
tagacaatccttaacccttaa

SEQ ID NO: 4 - DNA, 21 bp, Artificial Sequence
  siRNA
aagcatttatatcatcctcaa

SEQ ID NO: 5 - DNA, 21 bp, Artificial Sequence
  siRNA
tggaacaaactacaagtgcaa

SEQ ID NO: 6 - DNA, 21 bp, Artificial Sequence
  siRNA
aaagccctggatcatatgaaa

SEQ ID NO: 7 - DNA, 21 bp, Artificial Sequence
  siRNA
ctggttgtcagtctttagtaa

SEQ ID NO: 8 - DNA, 21 bp, Artificial Sequence
  siRNA
taggtggtcgtgtgatggtaa

SEQ ID NO: 9 - DNA, 21 bp, Artificial Sequence
  siRNA
ccggtgcaccatggaacttaa

SEQ ID NO: 10 - DNA, 21 bp, Artificial Sequence
  siRNA
cctgctaaagttagcttgtaa

SEQ ID NO: 11 - DNA, 21 bp, Artificial Sequence
  siRNA
ttcgacaatgctcacacccta

SEQ ID NO: 12 - DNA, 21 bp, Artificial Sequence
  siRNA
ctcaacacttactcaagacaa

SEQ ID NO: 13 - DNA, 21 bp, Artificial Sequence
  siRNA
cacgcccagcctgtagcatat

SEQ ID NO: 14 - DNA, 21 bp, Artificial Sequence
  siRNA
cagaccttatacgacatcgaa

SEQ ID NO: 15 - DNA, 21 bp, Artificial Sequence
  siRNA
aaacacggatttgccaccaaa

SEQ ID NO: 16 - DNA, 21 bp, Artificial Sequence
  siRNA
ctggctcttaatccttatgaa

SEQ ID NO: 17 - DNA, 21 bp, Artificial Sequence
  siRNA
cagagattacctggaaagtaa

SEQ ID NO: 18 - DNA, 21 bp, Artificial Sequence
  siRNA
cacacgtatttatatttggaa